(19) United States
(12) Patent Application Publication
(10) Pub. No.: US 2016/0271386 A1
(43) Pub. Date: Sep. 22, 2016

(54) ELECTROSTIMULATION IN TREATING CEREBROVASCULAR CONDITIONS

(71) Applicants: Ronnie Levy, Kochav-Yair (IL); Yiftach Beinart, Hod Hasharon (IL); Alon Shalev, Ra'anana (IL)

(72) Inventors: Ronnie Levy, Kochav-Yair (IL); Yiftach Beinart, Hod Hasharon (IL); Alon Shalev, Ra'anana (IL)

(21) Appl. No.: 14/658,433

(22) Filed: Mar. 16, 2015

(51) Int. Cl. A61N 1/05; A61N 1/08

(52) U.S. Cl. CPC A61N 1/05; A61N 1/08

(57) ABSTRACT

An electrostimulation device including an electrode shaft that includes a plurality of electrodes, a delivery device that includes a cannula, through which the electrode shaft is insertable, a fixation member fixable on the cannula, and a locking mechanism for selectively permitting and preventing relative movement between the electrode shaft and the delivery device.

### CROSS-REFERENCE TO RELATED APPLICATIONS

This application is a continuation of U.S. patent application Ser. No. 13/755,116, filed Jan. 31, 2013, which is incorporated herein by reference.

### FIELD OF THE INVENTION

The present invention relates generally to electro stimulation of receptors, such as chemoreceptors, baroreceptors and aortic arch receptors, such as for inducing changes in the diameter of blood vessels of the brain, including dilation and constriction.

### BACKGROUND OF THE INVENTION

The cardiovascular center of the brain includes groups of neurons scattered within the medulla of the brain stem, which regulate heart rate, contractility of the ventricles, and blood vessel diameter. The cardiovascular center receives input both from higher brain regions and from sensory receptors. The two main types of sensory receptors that provide input to the cardiovascular center are baroreceptors and chemoreceptors. Baroreceptors are pressure-sensitive sensory neurons that monitor stretching of the walls of blood vessels and the atria. Chemoreceptors monitor blood acidity, carbon dioxide level and oxygen level.

Outputs from the cardiovascular center flow along sympathetic and parasympathetic fibers of the autonomic nervous system. Sympathetic stimulation of the heart increases heart rate and contractility, whereas parasympathetic stimulation decreases heart rate. Thus autonomic control of the heart is the result of opposing sympathetic (stimulatory) and parasympathetic (inhibitory) influences. Autonomic control of blood vessels, on the other hand, is mediated exclusively by the sympathetic division of the autonomic nervous system.

The primarily function of chemoreceptors is to regulate respiratory activity. This is an important mechanism for maintaining arterial blood gases pO₂, pCO₂, and pH within appropriate physiological ranges. For example, a decrease in arterial pO₂ (hypoxemia) or an increase in arterial pCO₂ (hypercapnia) leads to an increase in the rate and depth of respiration through activation of the chemoreceptor reflex. Respiratory arrest and circulatory shock (which decrease arterial pO₂ and pH, and increase arterial pCO₂) dramatically increase chemoreceptor activity leading to enhanced sympathetic outflow to the heart and vasculature via activation of the vasomotor center in the medulla. Cerebral ischemia activates central chemoreceptors, which produces simultaneous activation of sympathetic and vagal nerves to the cardiovascular system.

The carotid bodies are located on the external carotid arteries near their bifurcation with the internal carotids. Each carotid body is a few millimeters in size and has the distinction of having the highest blood flow per tissue weight of any organ in the body. Afferent nerve fibers join with the sinus nerve before entering the glossopharyngeal nerve. A decrease in carotid body blood flow results in cellular hypoxia, hypercapnia, and decreased pH that lead to an increase in receptor firing. The threshold pO2 for activation is about 80 mmHg (normal arterial pO₂ is about 95 mmHg). Any elevation of pCO₂ above a normal value of 40 mmHg, or a decrease in pH below 7.4 causes receptor firing.

PCT Patent Application PCT/IL2012/000290, filed 2 Aug. 2012, describes stimulation of chemoreceptors and baroreceptors in a carotid artery. In one embodiment, a device is inserted intravascularly via the femoral artery. In another embodiment, a device is introduced in an extravascular approach.

### SUMMARY

The present invention seeks to provide further features to some of the devices described in PCT Patent Application PCT/IL2012/000290. The invention has many uses in the treatment of physiological disorders such as, but not limited to cerebral brain vasospasm, ischemia and brain injury. Embodiments of the invention can be used to stimulate the carotid sinus nerve, aortic nerve, chemoreceptors adjacent to the bifurcation of the carotid, baroreceptors adjacent to the bifurcation of the carotid, aortic arch chemoreceptors and aortic arch baroreceptors, and others, in order to induce changes in the diameter of blood vessels of the brain, including dilation and constriction.

### BRIEF DESCRIPTION OF THE DRAWINGS

The present invention will be understood and appreciated more fully from the following detailed description, taken in conjunction with the drawings in which:

FIGS. 1-1 and 1-2 are simplified illustrations of an electrostimulation device, constructed and operative in accordance with a non-limiting embodiment of the present invention;

FIG. 1-3 is a simplified illustration of an expandable member, useful in fixation of the device;

FIGS. 2-1 to 2-16 are simplified illustrations of a method of using the electrostimulation device, in accordance with a non-limiting embodiment of the present invention;

FIG. 3 is a simplified illustration of the electrostimulation device inserted in a neck of a patient with electrodes positioned at the carotid bifurcation, in accordance with a non-limiting embodiment of the present invention;

FIG. 4 is a simplified schematic illustration of dipole stimulation of the receptors or neurons, showing the electrical field around the electrodes;

FIGS. 5-1 to 5-3 are simplified illustrations of electrodes positioned at both sides of the carotid bifurcation, wherein all electrodes are collinear, in accordance with a non-limiting embodiment of the present invention;

FIGS. 6-1 to 6-4 are simplified illustrations of electrodes positioned at both sides of the carotid bifurcation in a three-dimensional pattern, in accordance with a non-limiting embodiment of the present invention; and

FIGS. 7-1 to 7-2 are simplified illustrations of electrodes are positioned lateral to the carotid bifurcation and parallel to the common carotid artery, in accordance with a non-limiting embodiment of the present invention.

### DETAILED DESCRIPTION

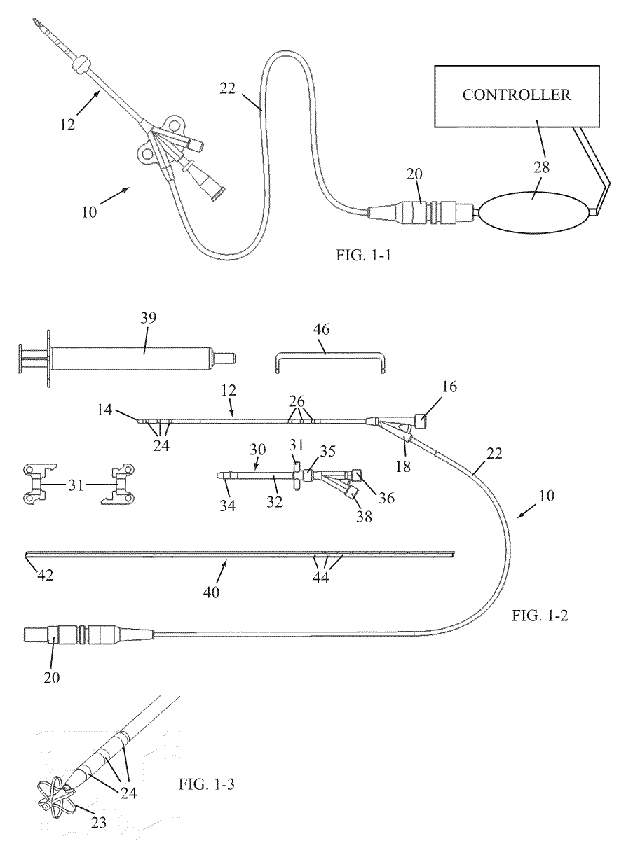

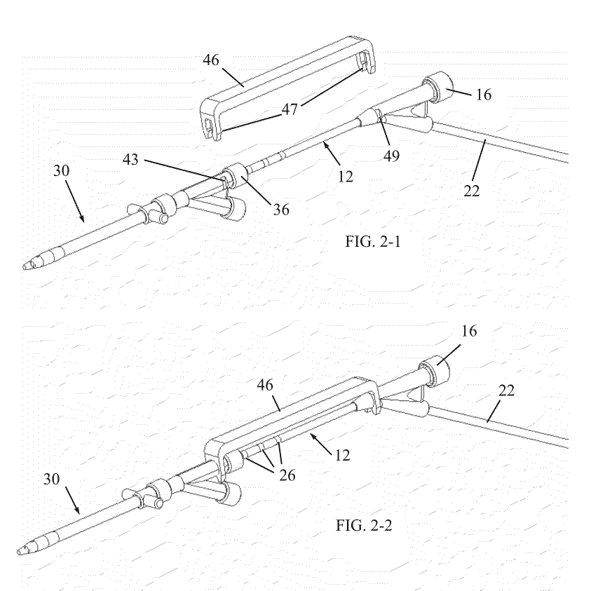

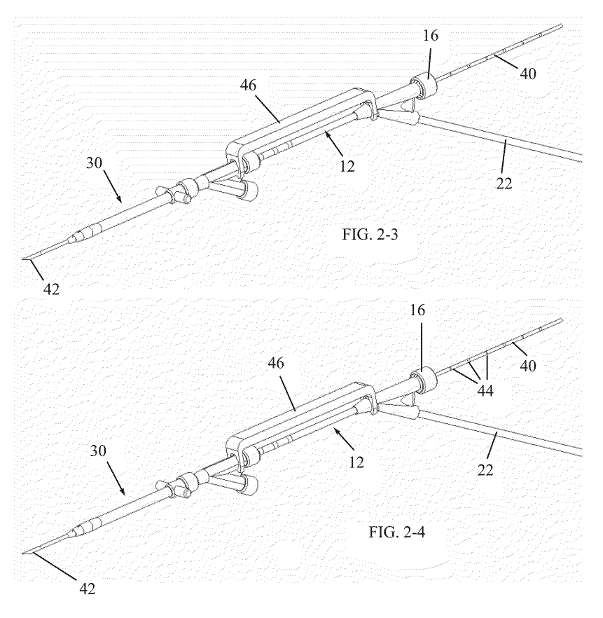

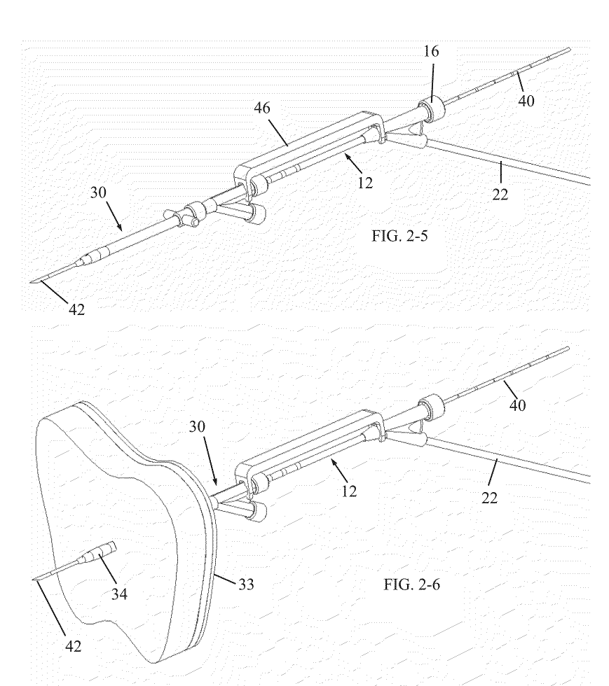

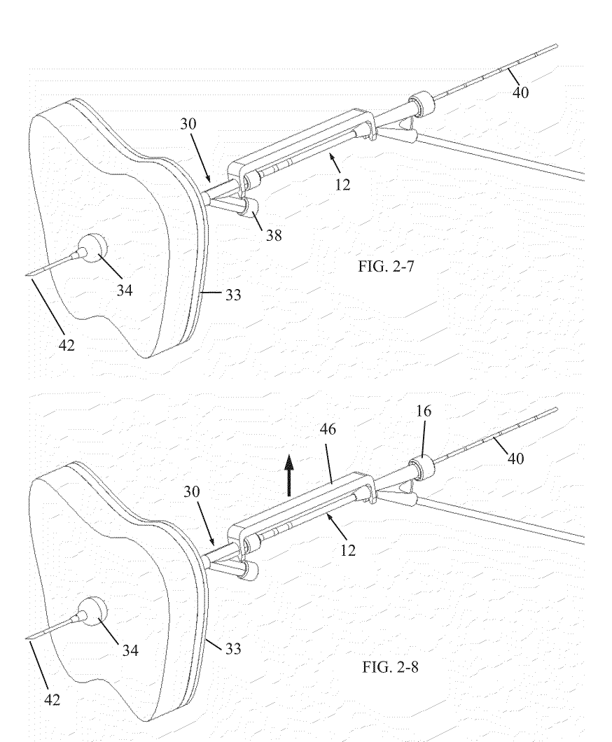

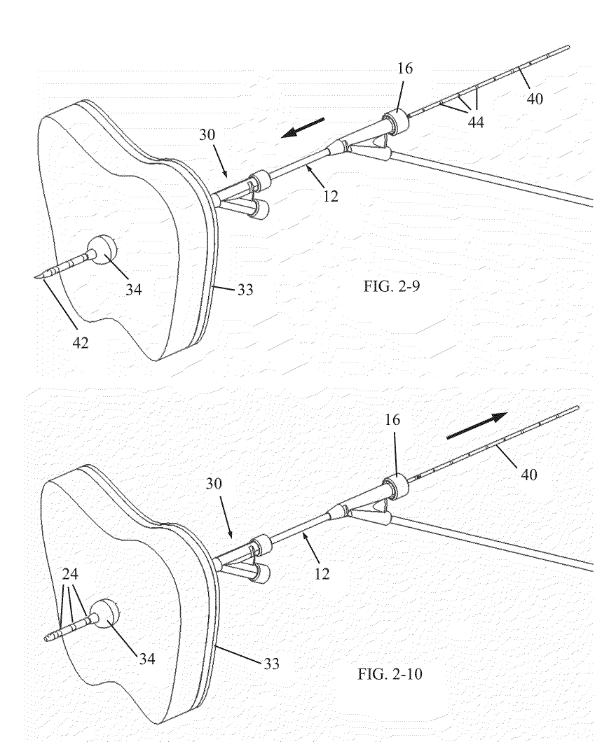

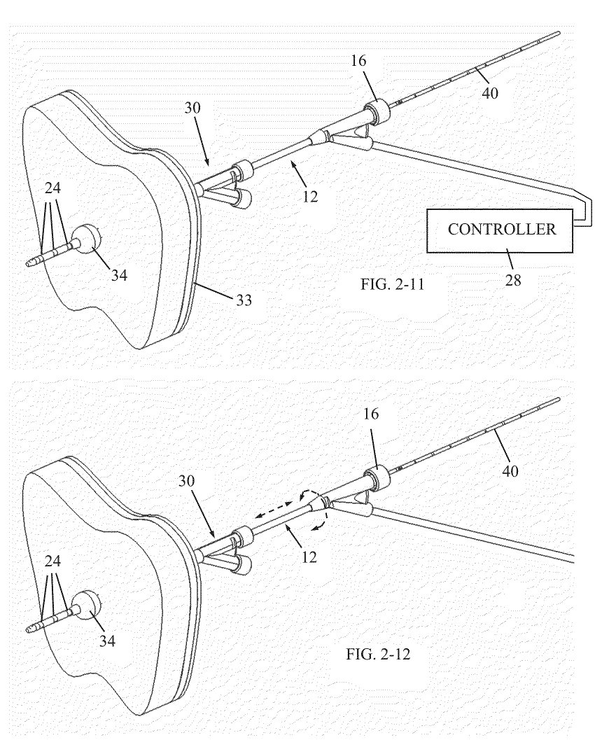

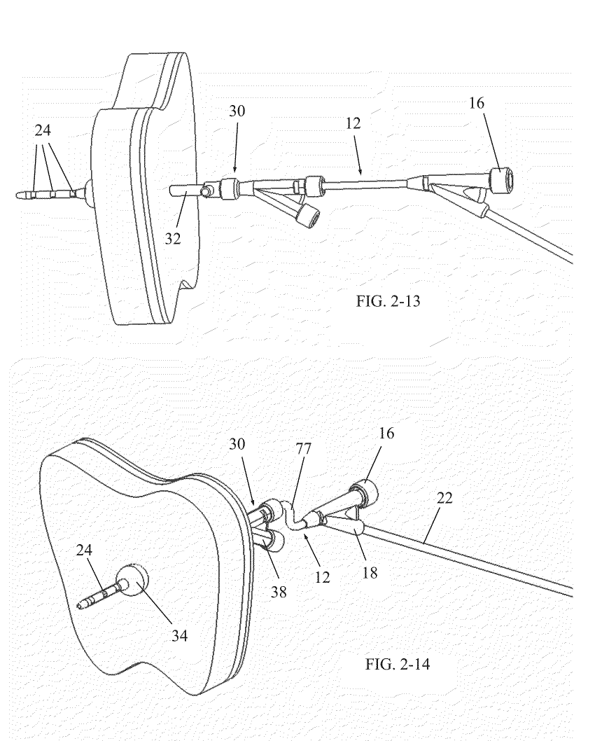

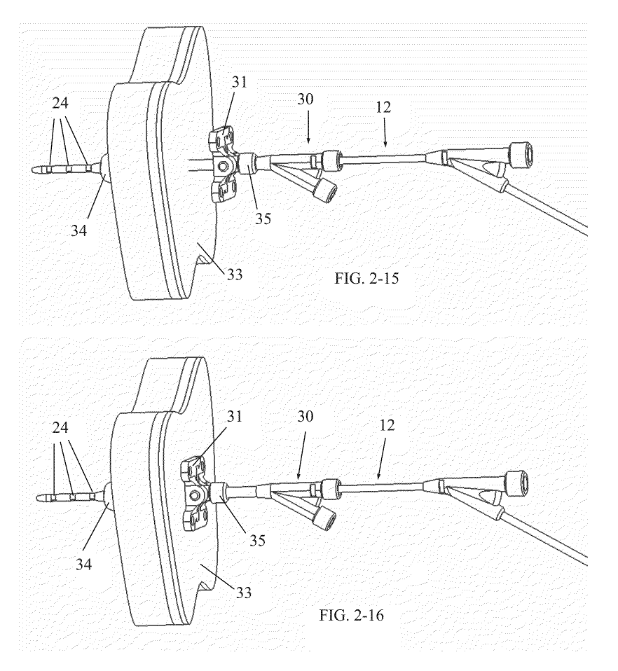

Reference is now made to FIGS. 1-1 and 1-2, which illustrate an electrostimulation device 10, constructed and operative in accordance with a non-limiting embodiment of the present invention. Electrostimulation device 10 includes an electrode shaft 12, which has a distal opening 14 and a proximal valve 16 plus one or more proximal branches 18, to which an electrical connector 20 is connected via a flexible cable 22. Electrode shaft 12 may include a plurality of axially spaced electrodes 24, such as near a distal portion thereof, which may be energized by an energy source (not shown). Electrodes 24 extend at least partially around a circumference of shaft 12. Thus in one embodiment, electrodes 24 are full 360° rings around shaft 12. In another embodiment, electrodes are partial rings that do not extend completely 360° around shaft 12. One or more fiducial markers 26, such as axially spaced stripes (which may be radiopaque), are proximal to the electrodes 24 (FIG. 1-2).

The electrical connector 20 is connected to a controller 28 (also called miniature autonomic unit 28, FIG. 1-1), which controls operating parameters associated with energization of electrodes 24, such as current and frequency of signals used to energize the electrodes.

The electric stimulation can be optimized by controller 28 and positioning the electrodes 24 relative to the target anatomy in order to achieve effective nerve stimulation and minimize side effects. These parameters control the shape and strength of the electrical field and its anatomic location. For example, current applied to the electrodes may be in, but is not limited to, the range of 0-10 mA. Voltage applied to the electrodes may be in, but is not limited to, the range of 0-25 V. The signals are preferably biphasic, but may be monophasic or a combination thereof. The distance between the effective electrodes can be in the range of about 1-20 mm, but the distance is not limited to this range.

The electrodes can be activated in any combination and in any order. The combinations and order can be changed during a stimulation session, either as part of a pre-determined sequence or in response to feedback from the patient.

The electrodes can range, without limitation, from about a tenth of a millimeter long to about 10 millimeter long. The electrodes can be cylindrical, partly-cylindrical with the base forming a sector of a circle, spherical, hemispheric, forming a section of a sphere, cylindrical with a polygonal base, cylindrical with a base forming a sector of a polygon, in the form of a triangular prism, in the form of a rectangular solid, in the form of an octahedral solid, in the form of a dodecahedral solid, in the form of an icosahedral solid, rectangular prism, ellipsoid, parallelepiped, star-shaped solid, helical and any combination thereof. Electrodes can be mounted longitudinally, transversely, or at an angle to supports.

The signal profile used to energize the electrodes can be of a wide variety—burst, prolonged, intermittent and any combination thereof. Individual groups of signals, such as but not limited to individual bursts, can have a step profile, a ramped profile that increases monotonically from the beginning to the end of the group of signals, a ramped profile that decreases monotonically from the beginning to the end of the group, a ramped profile which increases from a small value to a predetermined value, then remains constant until the end of the group, a ramped profile that starts at a predetermined value, remains at that value for a predetermined portion of the group, then decreases to a small value at the end of the group, a sinusoidal signal profile, a triangular signal profile, and any combination thereof.

The electrostimulation device 10 also includes a delivery device 30, which includes a cannula 32, which has a distal fixation member (which in this embodiment is a balloon) 34, a lockable proximal insertion port 36 and one or more proximal branch ports 38. A syringe 39, or other suitable fluid source, is provided for inflating balloon 34, such as through branch port 38 (also called inflation port 38) which may be in fluid communication with balloon 34. Delivery device 30 also includes an external fixation member 31 and a locking element or valve 35 (FIG. 1-2), distal to lockable proximal insertion port 36, also referred to as locking mechanism 36. As will be explained later, balloon 34 serves as an internal fixation member for fixation of the device in the patient. As seen in FIG. 1-3, instead of a balloon, other internal fixation members may be used, such as an expandable member 23 with loops that bend or buckle or otherwise deform outwards. The maximal axial cross-section of the internal fixation member (23 or 34) is increased in the deployment state of the device and decreased in the delivery state of the device. In one embodiment, the ratio of the maximal cross-sections of the internal fixation member (23 or 34) between the deployment and delivery states of the device is larger than 2. Fixation of the device is important, because even slight movement of the device may adversely affect treatment or even worse may cause harm to neighboring tissues. The external fixation member 31 may be a plate member with mounting holes for suturing.

The electrostimulation device 10 also includes a needle 40 with an echogenic distal tip 42 and a plurality of fiducial markers 44 proximal to tip 42.

The electrostimulation device 10 also includes a spacer 46, whose function will be described below.

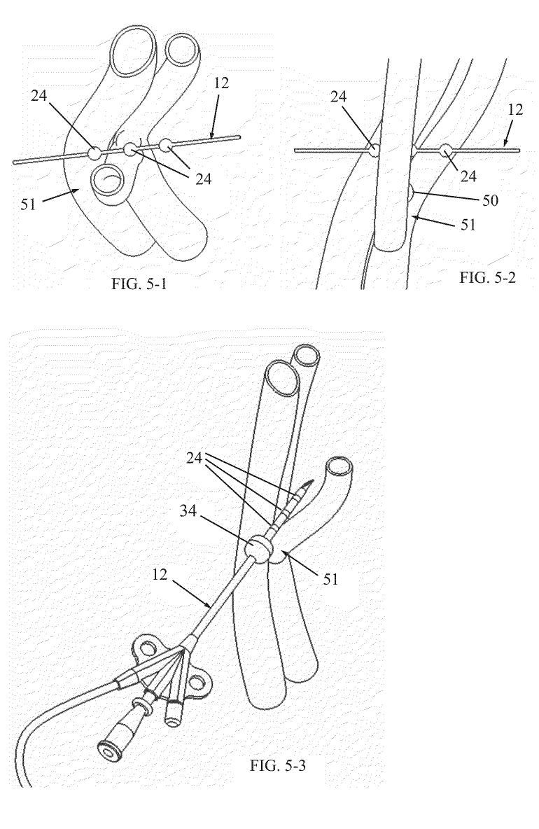

Reference is now made to FIGS. 2-1 to 2-16, which illustrate a method of using the electrostimulation device 10, in accordance with a non-limiting embodiment of the present invention.

Referring to FIG. 2-1, electrode shaft 12 is introduced through proximal insertion port 36 of delivery device 30. Spacer 46 is poised for positioning. In FIG. 2-2, spacer 46 is snapped, clamped or otherwise affixed to electrode shaft 12 and delivery device 30. For example, spacer 46 may be formed with a pair of notched ears 47 at opposite ends thereof (FIG. 2-1), one of which snugly fits into a groove 49 (FIG. 2-1) formed on a proximal head of electrode shaft 12 and the other of which snugly fits behind a collar 43 (FIG. 2-1) on delivery device 30. The affixed spacer 46 establishes a relative position of electrode shaft 12 with respect to delivery device 30. The electrode markers 26 on shaft 12 will serve as an indication for the amount of electrode exposure at the distal tip of delivery device 30, as explained later.

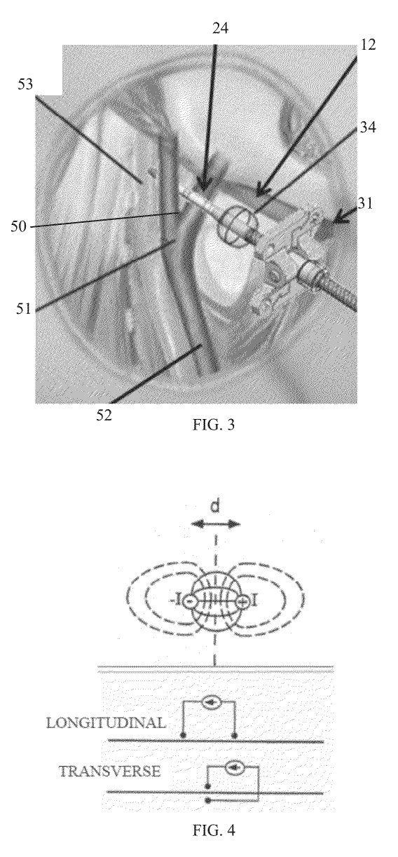

In FIG. 2-3, needle 40 is introduced through proximal valve 16 of electrode shaft 12 and passes all the way through delivery device 30, so that tip 42 of needle 40 extends out the distal end of delivery device 30. In FIG. 2-4, needle 40 is positioned axially to a desired position along electrode shaft 12 and delivery device 30, using fiducial markers 44 to indicate the axial position. In FIG. 2-5, proximal valve 16 of electrode shaft 12 is closed to lock needle 40 in place.

In FIG. 2-6, tip 42 of needle 40 punctures tissue 33, such as the tissue in a neck of a patient, for introducing the device to the carotid bifurcation (see FIG. 3). The assembly is passed through tissue 33 so that balloon 34 is on the inner side of the tissue wall. In FIG. 2-7, balloon 34 is inflated with fluid (e.g., saline) via inflation port 38, such as with the syringe 39 of FIG. 1-2. In FIG. 2-8, spacer 46 is removed.

In FIG. 2-9, the proximal valve 16 is unlocked so as to permit relative movement of shaft 12 with respect to needle 40. While holding needle 40 in place, electrode shaft 12 is moved distally until the proximal valve 16 moves past and just exposes a distal marker 44 of needle 40. Electrode shaft 12 now extends distally beyond the distal end of delivery device 30. As mentioned above, electrode markers 26 on shaft 12 serve as an indication for the amount of electrode exposure at the distal tip of delivery device 30. In FIG. 2-10, needle 40 is retracted slightly (if needed—until the most distal marker 44 is exposed) so that its distal tip does not protrude beyond electrode shaft 12. Proximal valve 16 is relocked.

In FIG. 2-11, electrical connector 20 is connected to controller 28 for operating electrodes 24. In FIG. 2-12, controller 28 is used to select and optimize stimulation parameters, such as but not limited to, voltage, frequency, pulse width, duty cycle and type of signals, used to energize the electrodes 24 (as mentioned more in detail above). In addition, the axial and radial orientation of the electrodes 24 may be optimized by unlocking locking mechanism 36 to allow radial and axial movement of shaft 12. In FIG. 2-13, after the optimization and orientation are done, the needle may be removed from electrode shaft 12. The assembly is now more flexible, because the needle is much more rigid than shaft 12 and cannula 32.

The flexibility of the assembly is now described with reference to FIG. 2-14. After removing the needle, electrode shaft 12 has a strain relief portion 77, which may be positioned between proximal branches 18 and electrodes 24. The strain relief portion 77 is flexible, and as seen in the drawing, can be bent to a curved shape (e.g., S-shape). The strain relief portion 77 significantly reduces any transfer of rotational torque and/or linear forces (push and/or pull forces) between the electrode shaft proximal valve 16 and branches 18 and the electrodes 24. This helps prevent disturbing the fixation of the device. Accordingly, without the needle, the strain relief portion 77 is considered to assume an active state, in which it is capable of reducing passage of rotational torque and linear forces. With the needle, the strain relief portion 77 is considered to assume a neutralized state, in which passage of rotational torque or linear forces is permitted (e.g., at least two fold higher than in its active state).

In FIG. 2-15, the external fixation member 31 is mounted on delivery device 30. In FIG. 2-16, external fixation member 31 is moved against tissue 33 and locking element 35 is secured against cannula 32. The external fixation member 31 is sutured to tissue 33.

Reference is now made to FIG. 3, which illustrates electrostimulation device 10 inserted in an extravascular approach through a neck of a patient, in accordance with a non-limiting embodiment of the present invention. Device 10 is inserted and positioned (as described above with reference to FIG. 2-12, so that the electrodes 24 are closely superior to the carotid bodies 50 near the carotid bifurcation 51, which is superior to the common carotid artery 52 and next to the internal jugular vein 53. The internal fixation balloon 34 and the external fixation member 31 are on opposite sides of the skin.

Electrostimulation of receptors, such as chemoreceptors, baroreceptors and aortic arch receptors, such as for inducing vasodilatation in blood vessels of the brain, is performed by energizing the electrodes 24 with the controller (also called electrical stimulation unit (ESU)) 28 (not shown in FIG. 3).

Dipole stimulation of the receptors or neurons is carried out by rapidly changing the electrical field around the electrodes 24, which is seen schematically in FIG. 4. The waveform of the electrical signal significantly affects the threshold of energy applied to the receptors. The longitudinal component of the electric field excites the nerve, so the current lines should be along the nerve's longitudinal axis; in other words, the electric field should be optimally implemented so that the longitudinal vectors are along the carotid body region. Balance biphasic waveforms are preferred because the equivalent charge is neutral and thus reduces possible tissue damage. The electric field should be localized and balanced as much as possible (longitudinally and radially) and its amplitude should be as low as possible in order to reduce possible side effects, such as other physiological effects, and tissue damage.

The following are non-limiting examples of position and orientation of electrodes for electrostimulation of the receptors.

In FIGS. 5-1 to 5-3, electrodes 24 are positioned at both sides of the carotid bifurcation 51, wherein all electrodes 24 are collinear, that is, along a single axis. This is the simple configuration of electrodes 24, which all lie on shaft 12.

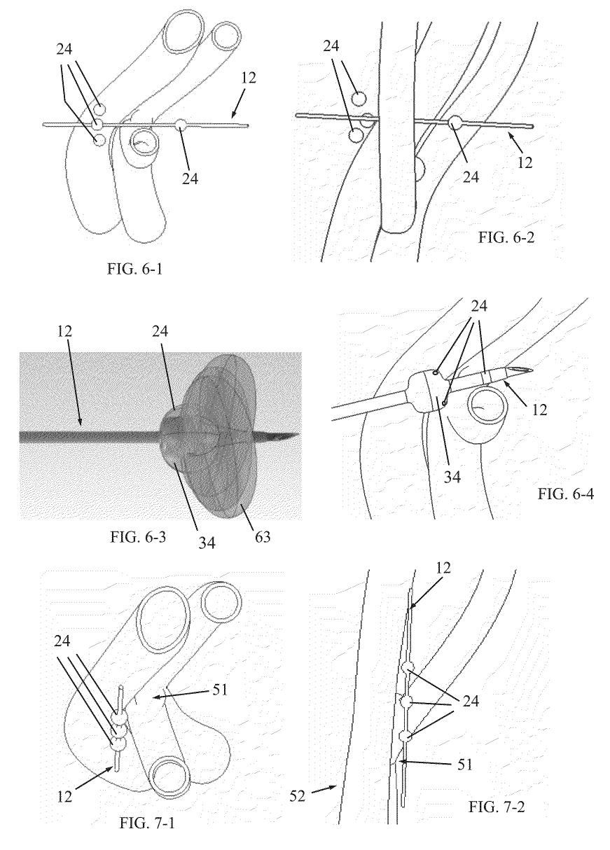

In FIGS. 6-1 to 6-4, one or more electrodes 24 are positioned on shaft 12 and one or more electrodes 24 are positioned on some inner deployed element, which may be a fixation member, either internal fixation balloon 34 or other expandable element or other structure. In this manner, the electrodes 24 are positioned at both sides of the carotid bifurcation 51 in a three-dimensional pattern. FIG. 6-3 shows possible 3D electrical fields 63 created by the electrodes 24, wherein the electrodes are not collinear but instead are positioned in different positions in 3D space. The electrodes can be positioned and energized in various manners to create many possible electrical fields.

In FIGS. 7-1 to 7-2, electrodes 24 are positioned lateral to the carotid bifurcation 51 and parallel to the common carotid artery 52. The electrodes 24 are collinear, that is, along a single axis.

1. An electrostimulation device comprising:
an electrode shaft that comprises a plurality of electrodes, said electrode shaft having a distal opening;
a proximal valve disposed on a proximal portion of said electrode shaft; and
a needle insertable through said proximal valve and said electrode shaft, wherein said needle is positionable axially to a desired position along said electrode shaft and a distal tip of said needle is positionable to extend out of said distal opening of said electrode shaft, and wherein closure of said proximal valve locks said needle with respect to said electrode shaft;
wherein said electrode shaft further comprises a strain relief portion capable of reducing transfer of rotational torque and linear forces to said electrodes, and wherein said strain relief portion has an active state, in which it is capable of reducing the transfer of rotational torque and linear forces, and a neutralized state, in which transfer of rotational torque or linear forces is permitted.

2. The electrostimulation device according to claim 1, wherein said electrodes are axially spaced from one another along said shaft and each electrode extends at least partially around a circumference of said shaft.

3. The electrostimulation device according to claim 1, wherein said shaft comprises one or more fiducial markers proximal to said electrodes.

4. The electrostimulation device according to claim 1, further comprising a controller in electrical communication with said electrodes, which controls operating parameters associated with energization of said electrodes.

5. The electrostimulation device according to claim 1, wherein said electrode shaft comprises an internal fixation member that comprises an expandable member, whose maximal axial cross-section is increased in a deployment state and decreased in a delivery state.

6. The electrostimulation device according to claim 1, wherein said needle comprises an echogenic distal tip.

7. The electrostimulation device according to claim 1, wherein one or more of said electrodes are positioned on said shaft and one or more of said electrodes are positioned on another structure of said device.

8. A method for electrostimulation comprising:
introducing at least one electrostimulation device of claim 21 into a neck of a patient near or at a site of a carotid bifurcation and energizing said electrodes to cause neurostimulation of the carotid bifurcation.

9. The method according to claim 8, wherein said electrode shaft comprises an internal fixation member and wherein introducing the electrostimulation device comprises:
introducing said needle through said proximal valve of said electrode shaft, so that said distal tip of said needle extends out of said distal opening of said electrode shaft, and closing said proximal valve to lock said needle in place;
puncturing tissue of the patient with said tip of said needle, and passing through said tissue so that said internal fixation member is on an inner side of said tissue;
moving said electrode shaft distally until at least some of said electrodes extend distally beyond said distal tip of said needle and are positioned near target receptors of the carotid bifurcation; and
affixing said electrostimulation device with said fixation member.

10. The method according to claim 9, further comprising selecting and optimizing stimulation parameters of said electrodes.

11. The method according to claim 8, further comprising removing said needle from the patient.

12. The method according to claim 8, comprising positioning said electrodes at both sides of a carotid bifurcation, wherein said electrodes are collinear.

13. The method according to claim 8, comprising positioning said electrodes at both sides of a carotid bifurcation in a three-dimensional pattern.

14. The method according to claim 8, comprising positioning said electrodes lateral to a carotid bifurcation and parallel to a common carotid artery.
